(19) United States
(12) Patent Application Publication
(10) Pub. No.: US 2020/0405959 A1
(43) Pub. Date: Dec. 31, 2020

(54) Planar Micro Injector With Securing Lanyard

(71) Applicant: Robert Carpenter, Kerrville, TX (US)

(72) Inventor: Robert Carpenter, Kerrville, TX (US)

(21) Appl. No.: 16/457,963

(22) Filed: Jun. 29, 2019

(51) Int. Cl. A61M 5/28; A61M 5/32

(52) U.S. Cl. CPC A61M 5/282; A45F 2005/006; A61M 5/329; A61M 5/3202

(57) ABSTRACT

A generally planar micro injector with a securing lanyard is disclosed. The micro injector is constructed of an elastic ampoule with a flexible actuation area affixed to a thin base unit through which an injector needle is plumbed. The micro injector is secured to the hand with an adhesive layer and an affixed securing lanyard.

### CROSS-REFERENCE TO RELATED APPLICATIONS

Not applicable.

### FIELD OF THE INVENTION

The present invention relates to a generally planar micro injector with a securing lanyard. The micro injector is constructed of an elastic ampoule with a flexible actuation area affixed to a thin base unit through which an injector needle is plumbed. The micro injector is secured via an affixed securing lanyard.

### BACKGROUND OF THE INVENTION

Currently, dentists and surgeons use injector devices with needles affixed at one end and a pressure appreciating button or actuator at the other end. These injectors are roughly linear and tubular and narrow in their construction. When operating in an enclosed space, such as the oral cavity, the needle must be inserted through the mouth and directed towards the area to receive the injection.

In the modern practice of dentistry, however, the fingers of the dentist are used to navigate the mouth of the patient. Therefore a first goal of the present invention is a micro injector roughly planar in shape that may be affixed to the end of one finger.

A second goal of the present invention is a securing lanyard or cord affixed to the micro injector and leading back into the hand.

A third goal of the present invention is a securing cover to protect the injector and prevent its actuation before the proper time.

A fourth goal of the present invention is a micro injector prefilled with a variety of anesthetics and antibiotics.

### SUMMARY OF THE INVENTION

The exemplary embodiment of the present invention comprises three components: 1) A generally circular, planar injector with a flexible actuation surface on one side and a perpendicularly aligned injector needle on the opposite side. Optionally, projecting out from the opposite side is a flexible ring surrounding the perpendicularly aligned needle. This ring is filled with an anesthetic gel; 2) A fiber, plastic, or nylon lanyard attached to a point on the circular outer periphery of the generally circular, planar injector; and, 3) A bilaterally distributed protective cover capable of securing the generally circular, planar injector and ensuring that the flexible actuation surface is not activated and thus the medication inside the micro injector remains inside and the perpendicularly aligned injector needle remains sterile until use. In those optional embodiments where a flexible ring surrounds the perpendicularly aligned needle and the ring is filled with an anesthetic gel, the protective cover also prevents the anesthetic gel from being exposed to the air.

The generally circular, planar injector ranges from about 1 cm to 3 cm in diameter and holds typically 0.2 cc of liquid medication. Those having skill in the art will recognize that different sizes of generally circular, planar injectors are cable of holding differing amounts of medication and that all such combinations are implicitly included in this patent disclosure.

The generally circular, planar injector is constructed of a base disc constructed of plastic, nylon, acetyl homopolymer (Delrin®), polyoxymethylene, and similar plastic materials with a centrally located mounting boss perpendicularly aligned at the center of the circular base.

The mounting boss is molded with a perpendicularly aligned needle bore that proceeds through one half of the mounting boss and funnels out into the upper half of the mounting boss with the upper half of the mounting boss being above the plane of the circular base and the lower half of the mounting boss being below the plane of the circular base.

In an alternative embodiment, the mounting boss is molded with a perpendicularly aligned needle bore that proceeds through one half of the mounting boss and proceeds into the upper half of the mounting boss with the upper half of the mounting boss being above the plane of the circular base and the lower half of the mounting boss being below the plane of the circular base. Molded or drilled perpendicularly to, and fluidically communicating with, the needle bore in the upper half of the mounting boss is at least one fluid feed passage.

In another alternative embodiment, a flexible ring extends from the base disc beyond the tip of the needle and is filled with anesthetic gel. Anesthetic gels are selected from the those that contain procaine (Novocain), lidocaine, and prilocaine. Those having skill in the art will recognize that other anesthetics may be used or included and these are implicitly included in this application.

Overarching the back side of the generally circular, planar injector is a flexible injector housing. The flexible injector housing is constructed of flexible nylon co-polyamide thermo-plastic elastomers (TPE) or similar elastic resin and is roughly spherical in radius with the plane formed by the intersecting base disc forming a spherical cap of the sphere. Those having skill in the art will recognize that other geometries may be conceived. For example, the flexible injector housing may be planar and resident at the top of a short cylindrical non-flexible segment which is in turn affixed to the flexible injector housing and the base disc.

Lining the inside of the flexible injector housing (and any other inflexible segments in communication with it) and the portion of the base disc is a polyvinyl chloride (PVC) or similar plastic containment layer. Those having skill in the art will recognize that the outside of the flexible injector housing may be coated with a semi-adhesive film allowing the device to be controllably affixed to the end of a finger. The semi-adhesive film may be protectively covered by a peel off plastic, nylon, or paper strip.

Ordinarily, inside the containment layer is a liquid anesthetic or antibiotic. If an anesthetic, ordinarily 0.1% to 5% lidocaine is supplied. Those having skill in the art will recognize that the medication may or may not include a vasoconstrictor such as adrenaline or epinephrine or an analog such as felypressin. Those having skill in the art will also recognize that any liquid medication may be included or substituted.

Into the aforementioned needle bore is inserted or screwed or pressure fit a tubular hypodermic needle. Ordinarily, a 30 gauge stainless steel needle is used, but those having skill in the art will recognize that any gauge needle may be used. The needle is at least 2 mm long, preferably 3 to 5 mm in length. Those having skill in the art will recognize that the length of the needle may vary depending on the application and that all lengths are implicitly included in this application.

Affixed to the circular edge of the base disc of the generally circular, planar injector is a floss holder tab. Affixed or inserted through the floss holder tab is a segment of dental floss or some other flexible lanyard. The dental floss or flexible lanyard is included so that the dentist may peel the protective paper cover off of the sticky adhesive surface of the generally circular, planar injector, secure the injector to the tip of the forefinger, and extend the dental floss or some other flexible lanyard into the closed hand. This doubly secures the generally circular, planar injector to the hand.

As mentioned before, the outside of the flexible injector housing may be coated with an adhesive film allowing the device to be controllably affixed to the end of a finger. The adhesive film may be protectively covered by a peel off plastic, nylon, or paper strip. Similarly, the surface of the generally circular, planar injector from which the mounting boss and the needle project may be protected by a plastic, nylon, or paper securing patch that is adhesively attached to the generally circular, planar injector and is peeled off before the generally circular, planar injector is used. If an embodiment of the invention has a flexible ring cylindrically surrounding the needle filled with anesthetic gel, this ring and anesthetic gel may also be covered by a plastic securing patch that is adhesively attached to the generally circular, planar injector.

### BRIEF DESCRIPTION OF THE DRAWINGS

FIG. 1 is a perspective drawing showing the generally circular, planar injector.

FIG. 2a is a cross-sectional diagram of a generally circular, planar injector showing a funnel mounted needle.

FIG. 2b is a cross-sectional diagram of a generally circular, planar injector showing a fluidically coupled channel mounted needle.

FIG. 3 is a plan drawing of the generally circular, planar injector from the needle protruding side.

### DETAILED DESCRIPTION OF THE PREFERRED EMBODIMENT

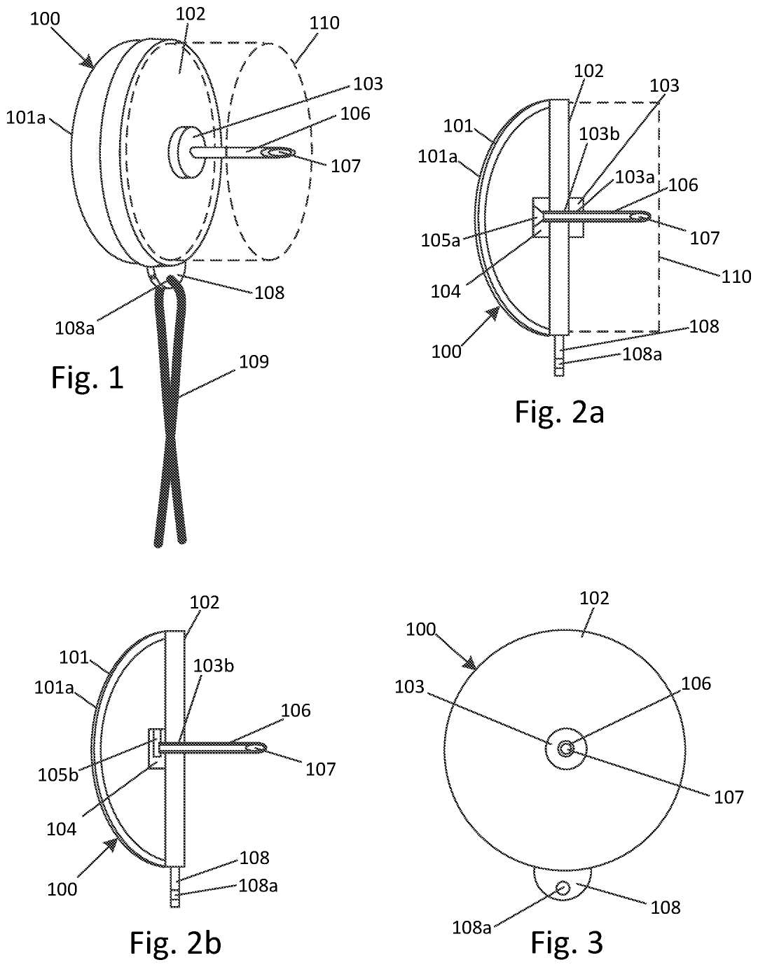

Referring now to FIGS. 1-3, the exemplary embodiment of the present invention comprises three components: 1) A generally circular, planar injector 100 with a flexible injector housing 101 on one side and a perpendicularly aligned injector needle 106 on the opposite side. Optionally, projecting out from the opposite side is a flexible ring 110 cylindrically surrounding the perpendicularly aligned needle 106. This flexible ring 110 is filled with an anesthetic gel. Anesthetic gels are selected from the family including benzocaine, procaine (Novocain), lidocaine, and prilocaine. Generally, benzocaine is present in the gel in about a 20% concentration, but may be present in a 0.1% to 35% concentration; 2) A fiber, plastic, or nylon lanyard 109 attached through a hole 108a on a tab 108 affixed to the circular outer periphery of base disc 102 of the generally circular, planar injector 100; and, 3) A bilaterally distributed, removable protective cover capable of securing the generally circular, planar injector 100 and ensuring that the flexible injector housing 101 is not activated and thus the medication inside the generally circular, planar injector 100 remains inside and the perpendicularly aligned injector needle 106 remains sterile and undamaged until use. In those optional embodiments where a flexible ring 110 cylindrically surrounds the perpendicularly aligned needle 106 and the flexible ring 110 is filled with an anesthetic gel, the protective cover also prevents the anesthetic gel from being exposed to the air.

The base disc 102 of the generally circular, planar injector 100 ranges from about 1 cm to 3 cm in diameter and holds typically 0.2 cc of medication. Those having skill in the art will recognize that different sizes of generally circular, planar injectors 100 are cable of holding differing amounts of medication and that all such combinations are implicitly included in this patent disclosure.

The generally circular, planar injector 100 is constructed of a circular base disc 102 constructed of plastic, nylon, acetyl homopolymer (Delrin®), polyoxymethylene, or similar plastic materials with a centrally located bottom half of mounting boss 103 perpendicularly aligned at the center of the circular base disc 102.

The bottom half of mounting boss 103 is molded with a perpendicularly aligned needle bore 103a that proceeds through the bottom half of the mounting boss 103. Contiguous segment of needle bore 103b proceeds through the circular base disc 102 and funnels out 105a into the upper half of the mounting boss 104 with the upper half of the mounting boss 104 being above the plane of the circular base disc 102 and the lower half of the mounting boss 103 being below the plane of the circular base disc 102.

In an alternative embodiment, the bottom half of mounting boss 103 is molded with a perpendicularly aligned needle bore 103a that proceeds through the bottom half of the mounting boss 103. Contiguous segment of needle bore 103b proceeds through the circular base disc 102 and into the upper half of the mounting boss 104 with the upper half of the mounting boss 104 being above the plane of the circular base disc 102 and the lower half of the mounting boss 103 being below the plane of the circular base disc 102. Molded or drilled perpendicularly to, and fluidically communicating with, the contiguous segment of needle bore 103b in the upper half of the mounting boss 104 is at least one fluid feed passage 105b.

In an alternative embodiment, mounting boss 103 is absent and perpendicularly aligned contiguous needle bore 103b proceeds through the circular base disc 102 and into the upper half of the mounting boss 104 with the upper half of the mounting boss 104 being above the plane of the circular base disc 102. Molded or drilled perpendicularly to, and fluidically communicating with, the needle bore 103b in the upper half of the mounting boss 104 is at least one fluid feed passage 105b.

Overarching the back side of the generally circular, planar injector 100 is a flexible injector housing 101. The flexible injector housing 101 is constructed of flexible nylon co-polyamide thermo-plastic elastomers (TPE) or some similar elastic resin and is roughly spherical in outer radius with the plane formed by the intersecting generally circular base disc 102 forming its spherical cap. Those having skill in the art will recognize that other geometries may be conceived. For example, the flexible injector housing 101 may be planar and resident at the top of a short cylindrical, non-flexible segment which is in turn affixed to the flexible injector housing 101 and the circular base disc 102.

Lining the inside of the flexible injector housing 101 (and any other inflexible segments in communication with it) and the portion of the generally circular base 102 that forms its base is a polyvinyl chloride (PVC) or similar plastic containment layer. The outside of the flexible injector housing 101 may be coated with an adhesive film 101a allowing the device to be controllably affixed to the end of a finger. The adhesive film 101a may be protectively covered by a peel off plastic, nylon, or paper strip.

Ordinarily, inside the plastic containment layer is a liquid anesthetic or antibiotic. Generally, procaine (Novocain) is present in about a 2% concentration, but various liquid anesthetics may be present in a 0.1% to 5% concentration. Those having skill in the art will recognize that the medication may or may not include a vasoconstrictor, such as adrenaline or epinephrine or an analog such as felypressin. Those having skill in the art will also recognize that any liquid medication may be included or substituted.

Into the aforementioned perpendicularly aligned needle bore 103a is inserted or screwed or pressure fit a tubular hypodermic needle 106. At the tip of the tubular hypodermic needle 106 is a beveled exit orifice 107. Ordinarily, a 30-gauge stainless steel tubular hypodermic needle 106 is used, but those having skill in the art will recognize that any gauge needle may be used. The tubular hypodermic needle 106 is at least 2 mm long, preferably 3 to 5 mm in length. Those having skill in the art will recognize that the length of the tubular hypodermic needle 106 may vary depending on the application and that all lengths are implicitly included in this application.

Affixed to tab 108 is a segment of dental floss or some other flexible lanyard 109. In the preferred embodiment, the dental floss or flexible lanyard 109 is passed through hole 108a in tab 108. The dental floss or flexible lanyard 109 is included so that the dentist may peel the protective plastic or vinyl cover off of the adhesive film 101a coating the flexible injector housing 101 and affix the generally circular, planar injector 100 to the tip of the forefinger and extend the dental floss or flexible lanyard 109 into the closed hand before using the generally circular, planar injector 100. This doubly secures the generally circular, planar injector 100 to the hand while it is being used.

As discussed supra, the outside of the flexible injector housing 101 may be coated with an adhesive film 101a allowing the device to be controllably affixed to the end of a finger. The adhesive film 101a may be protectively covered by a peel off plastic, nylon, or paper strip. Similarly, the surface of the circular base 102 from which the tubular hypodermic needle 106 projects may be protected by a plastic, nylon, or paper securing patch that is adhesively attached to the needle side of the generally circular, planar injector 100 and is peeled off before the generally circular, planar injector 100 is used. If an embodiment of the invention has a flexible ring 110 cylindrically surrounding the needle filled with anesthetic gel, flexible ring 110 and anesthetic gel may also be covered by a plastic securing patch that is adhesively attached to the generally circular, planar injector 100.

While the present invention has been described in what are thought to be the most useful and practical embodiments, it will be readily apparent to those having skill in the art that other variations may be readily conceived and created. Accordingly, these and all such other readily conceived and created variations are implicitly included in the spirit and scope of the present disclosure.

1. A generally circular, planar injector comprising:
a) a flexible injector housing mounted peripherally to a base;
b) wherein the base is penetrated by an outwardly projecting injector needle that is unmovably affixed to the base and the outwardly projecting injector needle is cylindrically surrounded by a flexible ring and the flexible ring is filled with anesthetic gel;
c) wherein the injector needle is fluidically coupled to a cavity formed between the flexible injector housing and the base;
d) wherein a floss lanyard is affixed to a floss tab at the periphery of the base;
e) wherein an [semi-]adhesive film is applied on the outer surface of the flexible injector housing and the flexible injector housing is controllably affixed to the end of a finger by means of the adhesive film; and
f) wherein a finger press against the flexible injector housing injects medicine from the cavity through and out of the outwardly projecting needle.

2. A generally circular, planar injector of claim 1 wherein the cavity formed between the flexible injector housing and the base is filled with liquid anesthetic.

3. A generally circular, planar injector of claim 2 wherein the liquid anesthetic is 2% lidocaine.

4. A generally circular, planar injector of claim 2 wherein the liquid anesthetic is 3% lidocaine.

5. A generally circular, planar injector of claim 2 wherein the liquid anesthetic is 5% lidocaine.

6. A generally circular, planar injector of claim 2 wherein the liquid anesthetic is 2% procaine.

7. A generally circular, planar injector of claim 2 wherein the liquid anesthetic is 3% procaine.

8. A generally circular, planar injector of claim 2 wherein the liquid anesthetic is 5% procaine.

9. A generally circular, planar injector of claim 2 wherein the liquid anesthetic contains a vasoconstrictor.

10. A generally circular, planar injector of claim 9 wherein the vasoconstrictor is epinephrine.

11. A generally circular, planar injector of claim 9 wherein the vasoconstrictor is felypressin.

12. A generally circular, planar injector of claim 1 wherein the injector needle is molded into the injector.

13. A generally circular, planar injector of claim 1 wherein the injector needle is screwed into the injector.

14. A generally circular, planar injector of claim 1 wherein the injector needle is pressure fit into the injector.

15. A generally circular, planar injector of claim 1 wherein the injector needle is a 30-gauge needle.

16. A generally circular, planar injector of claim 1 wherein the injector needle is at least 2 mm in length.

17. (canceled)

18. A generally circular, planar injector of claim 1 wherein the anesthetic gel contains benzocaine in a concentration selected from the range of about 0.1% to about 35% concentration.

19. A method of using a generally circular, planar injector comprising the steps of:
a) removing the removable protective cover to expose the injector needle;
b) removing a protective film to expose an adhesive surface on a flexible injector housing;
c) adhering the generally circular, planar injector to the tip of a finger;
d) extending the floss lanyard backward into the hand;
e) grasping the floss lanyard in the hand; and
f) injecting medication into the patient.

20. A method of using a generally circular, planar injector of claim 1 comprising the steps of:
a) removing the removable protective cover to expose the injector needle;
b) removing a protective film to expose an adhesive surface on a flexible injector housing;
c) adhering the generally circular, planar injector to the tip of a finger by means of the semi-adhesive film;
d) extending the floss lanyard backward into the hand;
e) grasping the floss lanyard in the hand; and
f) injecting medication into the patient.
